(19) United States
(12) Patent Application Publication
(10) Pub. No.: US 2026/0348980 A1
(43) Pub. Date: Nov. 5, 2026

(54) METHODS FOR DISCRIMINATING HEMORRHAGIC STROKE FROM ISCHEMIC OR MIMIC STROKE USING TPA AS BIOMARKER

(71) Applicants: INSTITUT NATIONAL DE LA SANTÉ ET DE LA RECHERCHE MÉDICALE, Paris (FR); CENTRE HOSPITALIER UNIVERSITAIRE DE CAEN, Caen (FR); UNIVERSITÉ DE CAEN NORMANDIE, Caen (FR)

(72) Inventors: Benoit ROUSSEL, Caen (FR); Audrey THIEBAUT, Boulogne Billancourt (FR); Denis VIVIEN, Caen (FR)

(21) Appl. No.: 19/486,665

(22) PCT Filed: May 22, 2024
(86) PCT No.: PCT/EP2024/064107

§ 371 Date: Nov. 21, 2025

(30) Foreign Application Priority Data
May 23, 2023 (EP) 23305817.1

(51) Int. Cl. G01N 33/68

(52) U.S. Cl. CPC G01N 33/6893; C12Y 304/21068; G01N 2333/9726; G01N 2800/2871

(57) ABSTRACT

Stroke is the second cause of death and the first cause of adult disability in industrialized countries. The goal of emergency therapy for acute ischemic stroke is prompt restoration of blood flow to regions of brain that are ischemic but not yet infarcted. Based on this, the inventors compare the plasma concentration of total tPA (antigenic: active+PAI-1 bound) in patients with acute ischemic stroke before any treatment with those in a healthy control group, to evaluate the interest of tPA as a potential diagnostic biomarker in the acute phase of ischemic stroke. In particular, the present invention relates to method for discriminating at early stage an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) concluding when the level of tissue-type plasminogen activator (tPA) determined at step i) is higher than the reference value is predictive of a risk of having or developing an ischemic stroke or a mimic stroke or concluding when the level of tissue-type plasminogen activator (tPA) determined at step i) is lower than the reference value is predictive of a risk of having or developing a hemorrhagic stroke

### FIELD OF THE INVENTION

The present invention is in the field of neurology. In particular, the invention provides methods and compositions for discriminating and treating stroke.

### BACKGROUND OF THE INVENTION

Stroke is the second cause of death and the first cause of adult disability in industrialized countries. The goal of emergency therapy for acute ischemic stroke is prompt restoration of blood flow to regions of brain that are ischemic but not yet infarcted.

In 1991, Collen and Lijnen used a cloned recombinant form of tissue-type plasminogen activator (tPA) to promote the lysis of blood clots by inducing the degradation of fibrin in a plasminogen-dependent manner (PMID 1742478). The Food and Drug Administration then approved it in 1996 to treat ischemic stroke in the United States (NINDS 1995) and it is so far the only approved acute pharmacological treatment. However, this treatment has some limitations, the main one being the short therapeutic window: it must be administrated within the first 4,5 hours following stroke onset (Thiebaut et al, Lancet Neurol 2018 PMID 30507392). Also, thrombolysis increases by 10 the risk of hemorrhagic transformation (NINDS 1995), and the risk is correlated to the time of injection from the onset.

The diagnosis of ischemic stroke is made with the association of clinical criteria (acute neurologic deficit) and cerebral imaging (Computerized Tomography scan or Magnetic Resonance Imaging). Even if a CT scan can be performed easily, its result is often normal at the acute phase. Magnetic resonance imaging is more sensitive to detect acute ischemia but could be difficult to perform due to its availability. In this context, the development of a biomarker is of great interest.

Endogenous tPA has already been studied as a biomarker in cardiovascular pathologies, in which an increase circulant rate was a marker of increase risk of event (Margaglione et al Arterioscler Thromb 1994). Interestingly enough, there is a linear relation between tPA and PAI-1 (plasminogen activator inhibitor 1, the main inhibitor of tPA) plasma concentrations. In fact, high concentrations of tPA are associated with high concentrations of PAI-1, resulting in low tPA activity (Olofsson BO Eur Heart J 1989; Oseroff A J Lab Clin Med 1989). In ischemic stroke, previous studies show an increase plasma tPA rate in patients with a history of stroke (Lindgren et al, Stroke 1996), however it is impossible to use this study as a diagnosis tool as plasma was harvested up to 7 days following the event. To defend the organism against intravascular thrombosis, endothelial cells acutely release tPA (Oliver et al Arterioscler Thromb 2005). In humans, experiments performed in the forearm showed that the release of tPA can be up to 4.5 μg/min and thus for many hours (Witherow et al, Journal of the American college of cardiology 2002). This is enough to reach concentrations similar to systemic thrombolysis. The fluid flow and the shear stress participate to the release of tPA by endothelial cells (Diamond et al, Science 1989). Interestingly, while a venous shear stress does not increase the release of tPA and PAI-1 (plasminogen activator inhibitor, type 1), an arterial shear stress increases their release 2.1 and 3 times respectively (Diamond et al, Science 1989).

Based on this, the inventors compare the plasma concentration of total tPA (antigenic: active+PAI-1 bound) in patients with acute ischemic stroke before any treatment, in patient with hemorrhagic stroke before any treatment, with those in a mimic stroke group, to evaluate the interest of tPA as a potential diagnostic biomarker in the acute phase of ischemic stroke or hemorrhagic stroke.

### SUMMARY OF THE INVENTION

Stroke is the leading cause of death and disability. Timely differentiation between ischemic stroke, hemorrhagic stroke, and stroke mimics is critical for tailored treatment and triage. To accelerate the identification of stroke's subtype, the inventor propose to use the levels of circulating tPA as a biomarker.

The present invention also relates to a method for discriminating at early stage an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) concluding when the level of tissue-type plasminogen activator (tPA) determined at step i) is higher than the reference value is predictive of a risk of having or developing an ischemic stroke or a mimic stroke or concluding when the level of tissue-type plasminogen activator (tPA) determined at step i) is lower than the reference value is predictive of a risk of having or developing a hemorrhagic stroke. In particular, the invention is defined by claims.

### DETAILED DESCRIPTION OF THE INVENTION

In the present invention, the inventors evaluated the level of tPA as a biomarker for identifying and discriminating the subtype of stroke, i.e. an ischemic stroke or a mimic stroke from or hemorrhagic stroke. In particular, they evaluated the expression level of total tPA on citrated plasma as biomarker of discrimination of stroke.

### Main Definitions

As used herein, the terms “subject” or “patient” denote a mammal, such as a rodent, a feline, a canine, and a primate. Particularly, the subject according to the invention is a human. Particularly, the subject according to the invention is a child, a teenager, an adult or an elderly person. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old. In a particular embodiment, the subject of the present invention did not receive any treatment (e.g. specific stroke treatment) before the methods of the present invention (i.e. assessment or diagnostic). In a particular embodiment, the subject of the present invention did not receive any treatment (e.g. specific stroke treatment) before the medical care. In some embodiment, the subject of the present invention did not receive any treatment (e.g. specific stroke treatment) before arriving at the hospital.

In some embodiment, the subject of the present invention is diagnosed or assessed for medical care by the methods of the invention in the ambulance, before arriving at the hospital or just at its arrival at the hospital or emergency department and before any treatment (e.g. specific stroke treatment). As used herein, the terms “protein” and “polypeptide” are used herein interchangeably, and refer to an amino acid sequence having more than 500 amino acids. As used herein, the term “protein” encompasses amino acid sequences having between 500 and 1000 amino acids, preferably between 510 and 900 amino acids, preferably between 520 and 800 amino acids, preferably between 525 and 700 amino acids.

As used herein, the term “tPA” or “tissue plasminogen activator” (abbreviated tPA for the protein or PLAT for the gene) has its general meaning in the art and refers to the serine protease EC 3.4.21.68. The tPA protein can be from any source, but typically is a mammalian tPA (e.g., human and non-human primate), particularly a human tPA. As used herein, the term “tPA” includes native tPA and recombinant tPA. tPA is commercially available as alteplase (Activase® or Actilyse®). Several analogues of tPA (such as deletion mutants) can also be used according to the invention; such analogues can be selected form the group of retaplase, tenekteplase, lanoteplase, monteplase, pamiteplase (Longstaff & Thelwell, FEBS Letters 579: 3303-9 (2005). The protein contains three potential sites for addition of N-linked oligosaccharides at Asn-117, Asn-184 and Asn-448, but is synthesized in mammalian cells as a mixture of two forms that differ in their extent of glycosylation (Pohl et al., 1984, Biochemistry, 23, 3701-3707). Type-1 tPA is glycosylated at all three sites, whereas type-2 tPA contains only two oligosaccharides, lacking side chain normally attached to Asn-184. These both forms of tPA can be used according to the present invention.

As used herein, the term “total tPA” encompass both forms of tPA: the single-chain tPA (sc-tPA), and cleaved tPA, also called two-chain tPA (tc-tPA).

As used herein, the term “sc-tPA” or “single-chain tPA” has its general meaning in the art and refers to the zymogen of tPA.

As used herein, the term “tc-tPA” or “two-chain tPA” has its general meaning in the art and refers to the cleaved form of tPA, obtained after cleavage of sc-tPA zymogen by plasmin at Arg278-Ile279.

As used herein, the term “enzyme able to cleave sc-tPA into tc-tPA” refers to any enzyme able to recognize the cleavage site of sc-tPA, described by analogy with other serine protease (Vehar et al., 1984), such as plasmin (Rijken et al., 1982), but also others enzymes such as kallikreins (Rajapakse et al., 2005) or factor Xa (Ichinose et al., 1984).

As used herein, the term “plasmin” has its general meaning in the art and refers to the active two-chain form of plasminogen, which is responsible for fibrinolysis. Plasminogen is the single-chain zymogen, precursor of plasmin. This zymogen is cleaved by tPA (or uPA) at Arg561-Val562 in human plasminogen. The plasminogen glycoprotein can be from any source, but typically is a mammalian plasminogen (e.g., human and non-human primate), particularly a human plasminogen.

As used herein, the term “stroke” has its general meaning in the art and refers to a medical condition in which poor blood flow to the brain causes cell death. There are two main types of stroke: ischemic which us due to lack of blood flow and hemorrhagic which is due to bleeding. Both cause parts of the brain to stop functioning properly. Signs and symptoms of a stroke may include an inability to move or feel on one side of the body, problems understanding or speaking, dizziness, or loss of vision to one side. Signs and symptoms often appear soon after the stroke has occurred. If symptoms last less than one or two hours, the stroke is a transient ischemic attack (TIA), also called a mini-stroke. A hemorrhagic stroke may also be associated with a severe headache. The symptoms of a stroke can be permanent. Long-term complications may include pneumonia and loss of bladder control.

As used herein, the term “ischemic stroke” relates to a medical condition where blood supply to part of the brain is decreased, leading to dysfunction of the brain tissue in that area. There are four reasons why this might happen: thrombosis (obstruction of a blood vessel by a blood clot forming locally), embolism (obstruction due to an embolus from elsewhere in the body), systemic hypoperfusion (general decrease in blood supply, e.g., in shock) or cerebral venous sinus thrombosis.

In some embodiment, the ischemic stroke can be an acute ischemic stroke (AIS) that is characterized by the sudden loss of blood circulation to an area of the brain.

As used herein, the term “hemorrhagic stroke” relates to a medical condition with two main types: Intracerebral hemorrhage, which is basically bleeding within the brain itself (when an artery in the brain bursts, flooding the surrounding tissue with blood), due to either intraparenchymal hemorrhage (bleeding within the brain tissue) or intraventricular hemorrhage (bleeding within the brain's ventricular system) and subarachnoid hemorrhage, which is basically bleeding that occurs outside of the brain tissue but still within the skull, and precisely between the arachnoid mater and pia mater (the delicate innermost layer of the three layers of the meninges that surround the brain).

As used herein, the term “mimic stroke” refers to a subject who arrives with a clinical picture that could suggest a stroke (hemiplegia, neurological sign, severe headache, confusion, neurological disorders, . . . ) but after imaging (MRI or scanner) which is not a stroke.

### Methods for Discriminating According to the Invention

The present invention also relates to a method for discriminating at early stage an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) concluding when the level of tissue-type plasminogen activator (tPA) determined at step i) is higher than the reference value is predictive of a risk of having or developing an ischemic stroke or a mimic stroke or concluding when the level of tissue-type plasminogen activator (tPA) determined at step i) is lower than the reference value is predictive of a risk of having or developing a hemorrhagic stroke.

The present invention also relates to a method of diagnosis at early stage an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) concluding when the level of tissue-type plasminogen activator (tPA) determined at step i) is higher than the reference value is predictive of a high risk of having or developing an ischemic stroke or a mimic stroke or concluding when the level of tissue-type plasminogen activator (tPA) determined at step i) is lower than the reference value is predictive of a high risk of having or developing a hemorrhagic stroke.

The present invention also relates to a method of diagnosis at early stage the risk of having or developing an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) administering to said subject a therapeutically effective amount of tPA or a classical stroke treatment when the level of tissue-type plasminogen activator (tPA) determined at step i) is higher than the reference value.

The present invention also relates to a method of diagnosis at early stage the risk of having or developing an hemorrhagic stroke from an ischemic stroke or a mimic stroke in a subject in need thereof, comprising the steps of i) determining in a blood sample obtained from the said subject the level of tissue-type plasminogen activator (tPA), ii) comparing the level determined in step i) with a reference value and iii) practicing a surgical procedure to remove accumulated blood and drain cerebrospinal fluid when the level of tissue-type plasminogen activator (tPA) determined at step i) is lower than the reference values used herein, the terms “sample” or “biological sample” refer to any sample obtained from a subject, such as a serum sample, a plasma sample, a urine sample, a blood sample, a lymph sample, or a tissue biopsy. In preferred embodiments, the fluid sample is a blood sample.

As used herein, the term “blood sample” means a whole blood sample obtained from a subject. In particular, the blood sample includes the serum sample and plasma sample.

As used herein, the term “control sample” refers to a sample (e.g. a blood sample) from healthy subject, or to a healthy sample of the subject.

As used herein, the term “mimic stroke sample” refers to a sample (e.g. a blood sample) from a subject who arrives with a clinical picture that could suggest a stroke (hemiplegia, neurological sign, severe headache, confusion, neurological disorders, . . . ) but after imaging (MRI or scanner) which is not a stroke.

As used herein, the term “citrated plasma” refers to a quantity plasma mix with citrate. The citrated plasma allows to detect the total form of tPA and is the gold standard for studying fibrinolysis and coagulation. In some embodiment, a tube of citrate is used to perform the method of the invention.

In some embodiment, the total tPA is measured. In some embodiment, the method of the present invention is performed on citrated plasma. In a particular, the total tPA is measured on citrated plasma.

In some embodiment, the citrate is added in a concentration of 1%, 1.5%, 2%, 2.5%, 3%, 3.5%, 4%, 4.5%, 5% in the tube test. In particular, the citrate is added in a concentration of 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5% or 5% in the tube test.

The present invention also relates to a method of detection of total tPA in a blood sample wherein the total tPA is measured on citrated plasma.

As used herein, the term “at early stage” refers to the medical care, prior to any treatment (e.g. stroke treatment) or diagnosis: in the ambulance or before arriving at the hospital or just on arrival at the hospital or at the emergency department. In some embodiment, “at the early stage” as defined by the invention, the subject did not receive any treatment (e.g. stroke treatment) or therapeutic care.

As used herein, the term “stroke treatment” refers to the classical treatment of stroke. Treatment for stroke depends on the type of stroke.

For a hemorrhagic stroke the treatment consists of practicing a surgical procedure to remove accumulated blood and drain cerebrospinal fluid.

For ischemic stroke, where a blood clot or blockage in the brain causes this type of stroke type, the stroke treatments include:
Clot-breaking drugs: Thrombolytic drugs can break up blood clots in the arteries of the brain, which still stop the stroke and reduce damage to the brain.
   Tissue plasminogen activator (tPA), or Alteplase IV r-tPA, is considered the gold standard in ischemic stroke treatment. This drug works by dissolving blood clots quickly. People who receive a tPA injection are more likely to recover from a stroke and less likely to have any lasting disability as a result of the stroke. It must be given within 3 to 4.5 hours after symptoms of a stroke begin.
Direct-acting oral anticoagulants (DOACs), Anticoagulants, Antiplatelet drugs, Statins, Blood pressure drugs Mechanical thrombectomy: During this procedure, a catheter is inserted into a large blood vessel inside the head. Then a device is used to pull the clot out of the vessel. This surgery is most successful if it is performed 6 to 24 hours after the stroke begins.
Stents: If the artery walls are found to be weakened, a procedure to inflate the narrowed artery and support the walls of the artery with a stent is done.
Surgery: In the rare instances that other treatments do not work, surgery can remove a blood clot and plaques from your arteries. This surgery may be done with a catheter. If the clot is especially large, a surgeon may open an artery to remove the blockage.

As used herein, the term “discriminating” refers to an action of distinguishing, processing, considering, selecting entities according to specific or distinctive criteria.

As used herein, the term “diagnosis” is a medical term for determining or identifying the development of a disease. In particular, the method of diagnostic of the present invention is associated with the determination of the level of tPA, in particular the level of total tPA on citrated plasma as biomarker of ischemic stroke or mimic stroke or hemorrhagic stroke.

As used herein, the term “predicting” means that the subject to be analysed by the method of the invention is allocated either into the group of subjects who will relapse, or into a group of subjects who will not relapse after a treatment.

The method is particularly suitable for assessing or diagnosing the duration of the overall survival (OS), progression-free survival (PFS) and/or the disease-free survival (DFS) of the ischemic stroke or mimic stroke or hemorrhagic stroke subject. Those of skill in the art will recognize that OS survival time is generally based on and expressed as the percentage of people who survive a certain type of ischemic stroke or mimic stroke or hemorrhagic stroke for a specific amount of time. Ischemic stroke or mimic stroke or hemorrhagic stroke statistics often use an overall five-year survival rate. In general, OS rates do not specify whether ischemic stroke or mimic stroke or hemorrhagic stroke survivors are still undergoing treatment at five years. DSF gives more specific information and is the number of people with a particular ischemic stroke or mimic stroke or hemorrhagic stroke. Also, progression-free survival (PFS) rates (the number of people who consequence of ischemic stroke or mimic stroke or hemorrhagic stroke, but their disease does not progress) include people who may have had some success with treatment, but the consequence of ischemic stroke or mimic stroke or hemorrhagic stroke has not disappeared completely.

As used herein, the term “score” refers to a value allowing to determine the prognosis of a subject suffering stroke. Typically, to estimate the prognosis ability of tPA, inventors estimate the survival for subjects in the BioStroke cohort. Next, they plot a ROC curve. The ROC curve with tPA is 0.9489, thereby indicating that the score is of interest to estimate patients' prognosis. ROC curve is a graphic representation of the relation existing between the sensibility and the specificity of a test, that allows the determination and the comparison of the diagnostic performances of several tests. Different index associating sensitivity (se) and specificity (sp) have been proposed. The most classic is that of Youden, which is 1 when the test is perfect. The exploration of this to the single cell analysis, cells with a score above the Youden index should be endowed with aggressive tumorigenic properties and convey a “poor prognosis”, while those with a PC1 score under the Youden index should be associated “good prognosis”.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

As used herein, the “the risk of having or developing an ischemic stroke or a mimic stroke or a hemorrhagic stroke” means that the patient to be analyzed by the method of the present invention is allocated either into the group of patients of a population having an elevated risk, or into a group having a reduced risk of having ischemic stroke or a mimic stroke or a hemorrhagic stroke. An elevated risk as referred to in accordance with the present invention, preferably, means that the risk of having ischemic stroke or mimic stroke or hemorrhagic stroke within a predetermined predictive window is elevated significantly (i.e. increased significantly) for a patient with respect to the average risk measured in a general population. A reduced risk as referred to in accordance with the present invention, preferably, means that the risk of having ischemic stroke or mimic stroke or hemorrhagic stroke within a predetermined predictive window is reduced significantly for a patient with respect to the average risk measured in the general population. Particularly, a significant increase or reduction of a risk is an increase or reduction or a risk of a size which is considered to be significant for diagnosis or assessing, particularly said increase or reduction is considered statistically significant. The terms “significant” and “statistically significant” are known by the person skilled in the art. Thus, whether an increase or reduction of a risk is significant or statistically significant can be determined without further ado by the person skilled in the art using various well known statistic evaluation tools

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of
a) providing a collection of blood samples from subject suffering from ischemic stroke or mimic stroke or a hemorrhagic stroke;
b) providing, for each blood sample provided at step a), information relating to the actual clinical outcome for the corresponding subject
c) providing a serial of arbitrary quantification values;
d) quantifying the cell density for each blood sample contained in the collection provided at step a);
e) classifying said blood samples in two groups for one specific arbitrary quantification value provided at step c), respectively: (i) a first group comprising blood samples that exhibit a quantification value for level that is lower than the said arbitrary quantification value contained in the said serial of quantification values; (ii) a second group comprising blood samples that exhibit a quantification value for said level that is higher than the said arbitrary quantification value contained in the said serial of quantification values; whereby two groups of blood samples are obtained for the said specific quantification value, wherein the blood samples of each group are separately enumerated;
f) calculating the statistical significance between (i) the quantification value obtained at step e) and (ii) the actual clinical outcome of the subjects from which blood samples contained in the first and second groups defined at step f) derive;
g) reiterating steps f) and g) until every arbitrary quantification value provided at step d) is tested;
h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 blood samples of 100 subjects. The 100 samples are ranked according to the cell density. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding stroke subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

In some embodiment, the method of the present invention is performed in vitro or ex vivo.

The level of tPA of the invention may be determined by using standard electrophoretic and immunodiagnostic techniques, including immunoassays such as competition, direct reaction such as immunohistochemistry, or sandwich type assays. Such assays include, but are not limited to, Western blots; agglutination tests; enzyme-labelled and mediated immunoassays, such as ELISAs; biotin/avidin type assays; radioimmunoassays; immune electrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

Standard methods for detecting the level of tPA are well known in the art. Typically, the step consisting of detecting the marker may consist in using at least one differential binding partner directed against the marker.

For example, the level of tPA can be determined using the specific antibody described in experimental section, ELISA assays (described in Delbroek L. et al., J Pharm Biomed Anal. 2013 Mar. 25; 76:49-58.; Henderson et al., 2015; Scott et al., 2017), each utilizing a sandwich-ELISA approach by capture with a total LRRK2 antibody, followed by detection with a specific pS935-LRRK2 antibody and SIMOA assay (Quanterix, digital ELISA technology). Also, for measuring total level of tPA, a sandwich ELISA approach can be used by capture with a total tPA antibody followed by detection with a total tPA antibody generated by Dr. Dario Alessi (University of Dundee's MRC) with Michael J. Fox Foundation (MJFF) support) and conventional ELISA assay detection (described in Delbroek L. et al., J Pharm Biomed Anal. 2013 March 25; 76:49-58).

As used herein, the term “binding partner directed against the marker” refers to any molecule (natural or not) that is able to bind the surface marker with high affinity. The binding partners may be antibodies that may be polyclonal or monoclonal, preferably monoclonal antibodies. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or radioactive molecule or a non-radioactive heavy metals isotopes to the antibody or aptamer, as well as indirect labelling of the probe or antibody by reactivity with a detectable substance. An antibody or aptamer of the invention may be labelled with a radioactive molecule by any method known in the art. More particularly, the antibodies are already conjugated to a fluorophore (e.g. FITC-conjugated and/or PE-conjugated).

The aforementioned assays may involve the binding of the binding partners (ie. antibodies or aptamers) to a solid support. The solid surface could a microtitration plate coated with the binding partner for the surface marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled.

In a particular embodiment, the expression level of tPA is performed by enzyme linked immunosorbent assay from Molecular Innovation (Human Total tPA ELISA Kit, Innovative Research).

In some embodiment, when the expression level of the tPA is higher than the predetermined reference value, it is considered that the subject has or is at a risk of having a ischemic stroke or a mimic stroke.

In some embodiment, when the expression level of the tPA is lower than the predetermined reference value, it is considered that the subject has or is at a risk of having a hemorrhagic stroke.

The person skilled in the art knows how to determine a predetermined reference value.

In a particular embodiment, the method according to the invention further comprises a step of classification of subject by an algorithm and determining whether a subject will have either an ischemic stroke or a mimic, either a hemorrhagic.

Typically, the method of the present invention comprises a) quantifying the expression level of the tPA in the blood sample; b) implementing a classification algorithm on data comprising the quantified of tPA levels so as to obtain an algorithm output; c) determining the probability that the subject have a stroke from the algorithm output of step b).

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF) selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO 2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO 2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

### The Method of Treatment of the Present Invention

A further object of the invention relates to a method for treating an ischemic stroke comprising administering a subject in need thereof with a therapeutically effective amount of tPA or other classical stroke treatment.

The present invention also relates to a method for treating an ischemic stroke in a subject in need thereof comprising the steps of:
a) providing a sample containing blood from the patient
b) detecting the level of tissue-type plasminogen activator (tPA)
c) comparing the level determined at stet b) with a reference value and if level determined at step b) is higher than the reference value, treating the subject with tPA or a classical stroke treatment.

The present invention also relates to a method for treating an ischemic stroke in a subject in need thereof comprising the following steps:
i) determining the score of tPA in a blood sample obtained from the subject who did not receive any treatment
ii) ii) comparing the score quantified at step i) with its predetermined reference value;
iii) iii) providing a conclusion on prognosis when the score of tPA is higher than their predetermined reference value; and
iv) iv) administering to said subject a therapeutically effective amount of tPA or other classical stroke treatment.

A further object of the invention relates to a method for treating a hemorrhagic stroke comprising a surgical procedure to remove accumulated blood and drain cerebrospinal fluid.

The present invention also relates to a method for treating a hemorrhagic stroke in a subject in need thereof comprising the steps of:
a) providing a sample containing blood from the patient
b) detecting the level of tissue-type plasminogen activator (tPA)
c) comparing the level determined at stet b) with a reference value and if level determined at step b) is lower than the reference value, treating the subject with a surgical procedure.

The present invention also relates to a method for treating a hemorrhagic stroke in a subject in need thereof comprising the following steps:
i) determining the score of tPA in a blood sample obtained from the subject who did not receive any treatment
ii) comparing the score quantified at step i) with its predetermined reference value;
iii) providing a conclusion on prognosis when the score of tPA is lower than their predetermined reference value; and
iv) treating to said subject with a surgical procedure.

In a particular embodiment, the subject is identified as having a stroke by performing the method as described above.

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “classical stroke treatment” refers to the treatment with tPA, direct-acting oral anticoagulants (DOACs), anticoagulants, antiplatelet drugs, statins or blood pressure drugs.

As used herein, the term “direct-acting oral anticoagulants” (DOACs) refers to anticoagulants highly effective, which require less monitoring and may reduce the risk of brain bleed when take, for stroke prevention. DOACs include but are not limited to Apixaban (Eliquis®), Dabigatran (Pradaxa®), Rivaroxaban (Xarelto®), Edoxaban (Savaysa™.

As used herein, the term “anticoagulants” has its general meaning in the art and refers to chemical substances that prevent or reduce coagulation of blood, prolonging the clotting time. Example of anticoagulant agents (defined herein as agents that inhibit blood clot formation) include, without limitation, specific inhibitors of thrombin, factor IXa, FXa, factor XIa, factor XIIa or factor VIIa, heparin and derivatives, Vitamin K antagonists (VKA), Non-VKA Anticoagulant agents” and anti-tissue factor antibodies. Non-VKA (Vitamin K Antagonists) anticoagulants includes non-VKA oral anticoagulants (NOAC) such as direct-oral anticoagulants (DOAC). Examples of specific inhibitors of thrombin include hirudin, bivalirudin (Angiomax®), argatroban, and lepirudin (Refludan®). Examples of heparin and derivatives include unfractionated heparin (UFH), low molecular weight heparin (LMWH), such as enoxaparin (Lovenox®), dalteparin (Fragmin®), tinzaparin (Innohep®), nadroparine (Fraxiparine® or Fraxodi®); and synthetic pentasaccharide, such as fondaparinux (Arixtra®). Examples of DOAC anticoagulants include rivaroxaban (Xarelto®), apixaban (Eliquis®), edoxaban (Lixiana®), and dabigatran (Pradaxa®). In one preferred embodiment, the anticoagulant is an inhibitor of FXa. “Vitamin K antagonists (VKA)” include, in a non-limitative manner, warfarin (Coumadin®), phenocoumarol, acenocoumarol (Sintrom®), clorindione, dicoumarol, diphenadione, ethyl biscoumacetate, phenprocoumon, phenindione, fluindione (Previscan®) and tioclomarol.

As used herein, the term “antiplatelet drugs” also known as a platelet agglutination inhibitor or platelet aggregation inhibitor, is a member of a class of pharmaceuticals that decrease platelet aggregation and inhibit thrombus formation. They are effective in the arterial circulation where anticoagulants have little effect.

As used herein, the term “statins” also known as HMG-COA reductase inhibitors, are a class of lipid-lowering medications that reduce illness and mortality.

As used herein, the term “blood pressure drugs” refers to drugs used to treat high blood pressure. The classes of blood pressure medications include but are not limited to diuretics, Beta-blockers, ACE inhibitors, Angiotensin II receptor blockers, Calcium channel blockers, Alpha blockers, Alpha-2 Receptor Agonists, Combined alpha and beta-blockers.

As used herein, the term “surgical procedure” relates to a medical procedure involving an incision with instruments; performed to repair damage or arrest disease in a living body.

As used herein, the term “preventing” intends characterizing a prophylactic method or process that is aimed at delaying or preventing the onset of a disorder or condition to which such term applies.

As used herein the terms “administering” or “administration” refer to the act of injecting or otherwise physically delivering a substance as it exists outside the body into the subject, such as by mucosal, intradermal, intravenous, subcutaneous, intramuscular delivery, transdermal, local or rectal administration and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention. For example, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In some embodiments, the patient is administered with a pharmaceutical composition comprising the therapeutically effective amount of tPA as active principle and at least one pharmaceutically acceptable excipient.

As used herein the term “active principle” or “active ingredient” are used interchangeably. As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

The pharmaceutical compositions of the invention may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical compositions of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active substances in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The tPA composition of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, tPA may also be administered in a form of liposomes, echogenic liposomes or liposomes attached to the surface of microbubbles. Particularly, the combination of said substance and tPA may be administered in form of liposomes, echogenic liposomes or liposomes attached to the surface of microbubbles.

In a more particular embodiment, said substance administered in a form of liposomes or liposomes attached to the surface of microbubbles in combination with tPA is plasmin.

### Kit of the Present Invention

A further object of the invention relates to a kit suitable to assess or diagnose or classify the risk of having or developing an ischemic stroke or hemorrhagic stroke or a mimic stroke at early stage in a subject in need thereof.

Accordingly, the invention relates to a kit for use in the method for assessing or diagnosing or classifying the risk of having or developing an ischemic stroke or hemorrhagic stroke or a mimic stroke at early stage said kit comprising a reagent that specifically reacts with tPA and instructions to perform the predicting method as described above.

The invention will be further illustrated by the following figures and examples. However, these examples and figures should not be interpreted in any way as limiting the scope of the present invention.

### FIGURES

FIG. 1: Plasma levels of tPA. Comparison of plasma tPA values between hemorrhagic stroke, ischemic stroke and stroke mimics. (A) Box and whisker plots with min., max., 25th, 50th (median) and 75^{th} percentiles. Each circles represent a value for one tPA measurement. (B-D) Sample size and plasma levels of antigenic tPA comparison between hemorrhagic stroke versus ischemic stroke; hemorrhagic stroke versus stroke mimics; and ischemic stroke versus stroke mimics. Data are presented as median (IQR) Mann-Whitney U test. IQR=InterQuartile Range. CI: Confidence Interval. ns =not significant. ***P<0.001.

FIG. 2: Discrimination of a hemorrhagic stroke from an ischemic stroke.

FIG. 3: Discrimination of a hemorrhagic stroke from a mimic stroke or a ischemic stroke.

FIG. 4: Model A logistic regression and model H logistic regression of hemorrhagic stroke versus ischemic stroke and stroke mimics patients. (A) ROC curves representation for model A logistic regression with or without tPA levels. Logistic regression model without tPA (AUC=0.67—dotted line) and with tPA level (AUC=0.78—continuous line). (B) Logistic regression plot of odds ratios and 95% CI; model A (in black) and model A with tPA (in white). (C) ROC curves representation for model H logistic regression with or without tPA levels. Logistic regression model without tPA (AUC=0.75—dotted line) and with tPA level (AUC=0.86—continuous line). (D) Logistic regression plot of odds ratios and 95% CI; model H (in black) and model H with tPA (in white).

FIG. 5: Model A logistic regression and model H logistic regression of ischemic stroke versus hemorrhagic stroke and stroke mimics patients. (A) ROC curves representation for model A logistic regression with or without tPA levels. The logistic regression model without tPA (AUC=0.71—dotted line) and with tPA level (AUC=0.71—continuous line). (B) Logistic regression plot of odds ratios and 95% CI; model A (in black) and model A with tPA (in white). (C) ROC curves representation for model H logistic regression with or without tPA levels. Logistic regression model without tPA (AUC=0.65—dotted line) and with tPA level (AUC=0.65—continuous line). (D) Logistic regression plot of odds ratios and 95% CI; model H (in black) and model H with tPA (in white).

FIG. 6: Model A logistic regression and model H logistic regression of stroke mimics versus hemorrhagic and ischemic stroke patients. (A) ROC curves representation for model A logistic regression with or without tPA levels. Logistic regression model without tPA (AUC=0.84—dotted line) and with tPA level (AUC=0.84—continuous line). (B) Logistic regression plot of odds ratios and 95% CI; model A (in black) and model A with tPA (in white). (C) ROC curves representation for model H logistic regression with or without tPA levels. The logistic regression model without tPA (AUC=0.79—dotted line) and with tPA level (AUC=0.81—conitnuous line). (D) Logistic regression plot of odds ratios and 95% CI; model H (in black) and model H with tPA (in white).

### EXAMPLE

### Material & Methods

### Study Design and Patient Selection:

Patients of the study are from a biological collection named Biostroke. Biostroke is an observational, prospective, monocentric study performed at the emergency department of Centre Hospitalo-universitaire de Caen Normandie, France since 2018. Biostroke collection was approved by ethics committees and there is an assignment agreement between our Inserm unit and EFS (Etablissement Français du Sang; agreement n°PLER-UPR/2018/2017). All patients or relatives receive information at inclusion.

Biostroke includes all patients at their arrival at the emergency service with an age >18; with a transitory focal neurological deficit or persisting at the time of emergency room arrival which may correspond to a stroke or acute headache which can correspond to subarachnoid hemorrhage; with a time from symptom onset to blood samples collection ≤48 hours; and who require a biological exam. Patients with a history of chronicle inflammatory pathology, cancer or hemostasis trouble were excluded. After inclusion, clinical and radiological data were collected into standardized forms at hospital admission. All stroke diagnosis were reviewed and confirmed by a trained neurologist (EL).

Control group are from the EFS collection of Caen, which contains samples from healthy blood donors that gave their consent for research.

For the present study, we include all patients of the Biostroke study with a final diagnosis of ischemic stroke and all patients from EFS collection who gave their consent for research. Symptom's severity in the patient group was assessed with the National Institutes of Health Stroke Scale score.

### Blood Samples Collection and Biomarker Measurement:

Blood was drawn on admission at the emergency department before any treatment. Samples are constituted from remaining blood in citrate tubes sampled to routine hemostasis exam of patients admitted with a suspected stroke diagnosis. Blood samples are first centrifuged at 2300 g for 11 minutes at room temperature then a second time after a passage in the automat, with the same parameters. Plasma is collected for the routine exam of patient and the rest is frozen at −80 °C in the biobank until biomarker measurement. For the EFS samples, blood was collected in the morning (between 9 am and 12 am) in citrated tubes, and processed with the same protocol than Biostroke samples.

Plasma antigenic tPA measurement was performed by enzyme linked immunosorbent assay from Molecular Innovation (Human Total tPA ELISA Kit, Innovative Research). All assays were performed in duplicate according to the manufacturer's instructions.

### Models and Logistic Regression Construction

For our analysis, we designed two models: the ‘Ambulance model’ (referred to as model A); and the ‘Hospital model’ (referred to as model H). The construction of these models is as follows:
For model A, we consider age, sex, RACE score, and including or not tPA levels in the plasma.
For model H, we include age, sex, baseline NIHSS score, systolic blood pressure (SBP), and including or not tPA levels in the plasma.”

We assessed the efficacy of both models in distinguishing each group (i.e. hemorrhagic strokes, ischemic strokes, and stroke mimics) against the rest (i.e. one-versus-rest approach).

### Statistical Analysis:

All univariate analyses were conducted using RStudio, version 4.1.2. Categorical variables were presented as numbers (percentages), while continuous variables were expressed as mean±SD. We performed comparisons between each group, utilizing the χ2 test for categorical variables and, depending on the data distribution assessed by the Shapiro-Wilk test, either the Student t-test or the Mann-Whitney U test for continuous variables. A significance level of P<0.05 was considered statistically significant. To estimate the minimal sample size to detect the presence of tPA difference, we conducted a priori power analysis. This analysis revealed that a N=25 was sufficient to detect group differences in the level of tPA with a statistical power of 80%. G*Power (version 3.1.9.7) was utilized for these power calculations. The utility of tPA as a potential biomarker was assessed by comparing the area under the curve (AUC) of our logistic regression models. The rationale of the prespecified models was to adjust for known predictors of stroke. Those included age (continuous), sex (yes or no), baseline RACE score (continuous) for the “ambulance model”; and age (continuous), sex (yes or no), baseline NIHSS score (continuous), and systolic blood pressure (continuous) for the “hospital model”. These two models are detailed in the result section. Statistical comparison of AUC values was performed by generating model predictions through bootstrapping and computing corresponding AUC values 5000 times. Model predictions were additionally computed using a 10-fold cross-validation with 3 repetitions and stratified sampling. These analyses were performed using a combination of glmfit.m, perfcurve.m and cvpartition.m in Matlab (the MathWork).

### Results

### Patient Selection

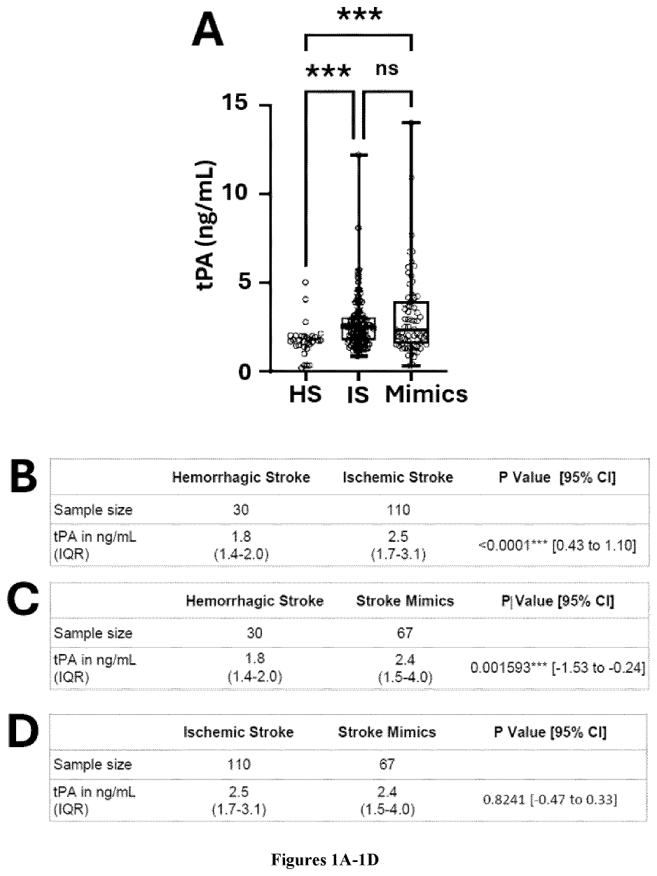

A total of 207 patients were enrolled in this study from 2018 to January 2022, originating from the Biostroke study conducted at Caen Normandie University Hospital in France. Among these individuals, 110 were confirmed to have experienced an ischemic stroke, 30 were confirmed to have had a hemorrhagic stroke, and 67 presented with conditions that mimicked stroke symptoms (FIG. 1). Of the total stroke patients (n=140), 79% were diagnosed with ischemic strokes (n=110), while 21% had hemorrhagic strokes (n=30; 27 (90%) ICH and 4 (10%) SAH); and in the entire cohort of included patients (n=207), 32% were categorized as stroke mimics (n=67). Importantly, the median time of sampling (from onset to sampling) was 1.3 hours.

### Measures of Plasma tPA Levels

The plasma levels of total antigenic tPA, assessed within the initial minutes of arrival at the emergency department, revealed noteworthy distinctions exclusively in the context of hemorrhagic strokes, as opposed to both ischemic strokes and stroke mimics (FIG. 1A). Hemorrhagic stroke patients display lower levels of plasma tPA compared to both ischemic stroke patients (1.8 ng/mL vs. 2.5 ng/ml, p<0.0001, 95% CI [0.43 to 1.1]; FIG. 1B) and stroke mimics (1.8 ng/mL vs. 2.4 ng/ml, p=0.001593, 95% CI [−1.53to −0.24]; FIG. 1C). Conversely, no significant differences were observed between ischemic stroke patients and stroke mimics (2.5 ng/mL vs. 2.4 ng/ml, p=0.8241, 95% CI [−0.47 to 0.33]; FIG. 1D). Given that the majority of our patients were admitted promptly after symptom onset (with a median time of 1.3 hours), we examined whether there was a correlation between tPA levels and the “symptom onset to hospital arrival” (Data not shown). However, our analysis did not reveal any significant association between plasma tPA levels and the time of arrival across all patient groups.

### Identification of Hemorrhagic Strokes Within the Cohort

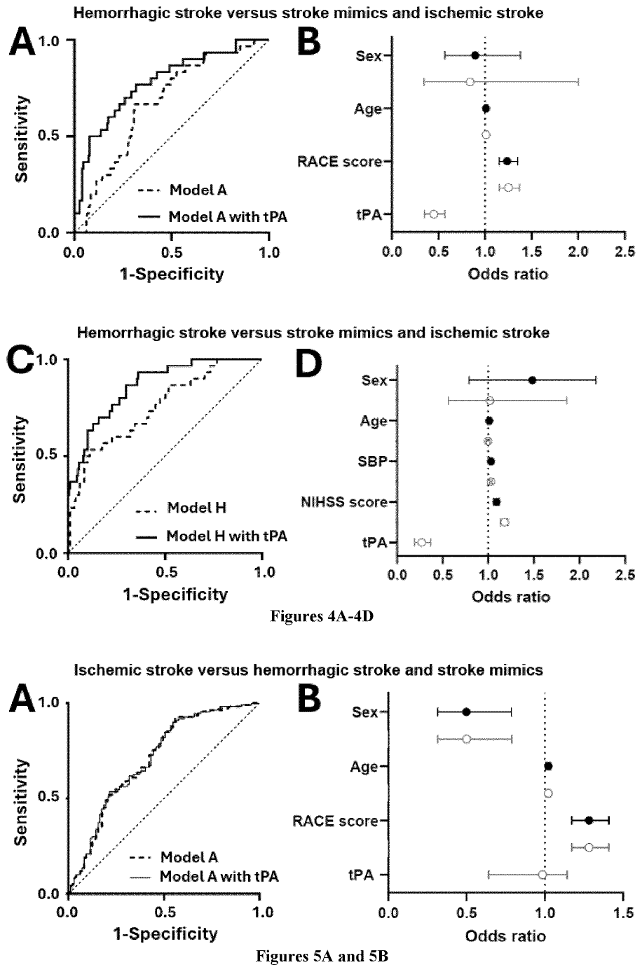

We first tested if plasma tPA levels could increase the accuracy to detect hemorrhagic strokes. The logistic regression model A incorporates sex, age, RACE score, and the presence or not of tPA plasma levels (FIG. 4A). In this model, tPA plasma levels (OR 0.45, 95% CI [0.35 to 0.57]) and RACE score (OR 1.25, 95% CI [1.15 to 1.37]) were significantly related to clinical status (FIG. 4B and data not shown). The accuracy of the model excluding tPA as abiomarker was 0.67, 95% CI [0.59 to 0.75]. Model accuracy significantly increased to 0.78, 95% CI [0.70 to 0.84] with the inclusion of tPA levels as a predictor (p=0.0098; FIG. 4A and data not shown). Critically, such increase in the model classification accuracy was further confirmed after the internal validation of these results through a 10-fold cross validation: 0.63, 95% CI [0.54 to 0.73] without tPA and 0.75, 95% CI [0.65 to 0.84] with tPA (p=0.0088; Data not shown).

Model H includes sex, age, SBP, baseline NIHSS, and the presence or not of tPA plasma levels (FIG. 4C). In this model, SBP (OR 1.03, 95% CI [1.02 to 1.04]), baseline NIHSS (OR 1.18, 95% CI [1.13 to 1.22]) and tPA levels (OR 0.27, 95% CI [0.19 to 0.37]) exhibited a significant relationship to the clinical status (FIG. 4D and data not shown). Under these conditions, the model's accuracy also improved, increasing from 0.75(95% CI [0.67 to 0.83]) without tPA to 0.86(95% CI [0.81 to 0.91]) with tPA (p=0.0024, FIG. 4C and data not shown). Furthermore, the 10-fold cross-validation accuracy increased from 0.72 (95% CI [0.61 to 0.81]) without tPA to 0.82 (95% CI [0.73 to 0.89]) with tPA (p=0.0196; Data not shown).

### Identification of Ischemic Strokes Within the Cohort

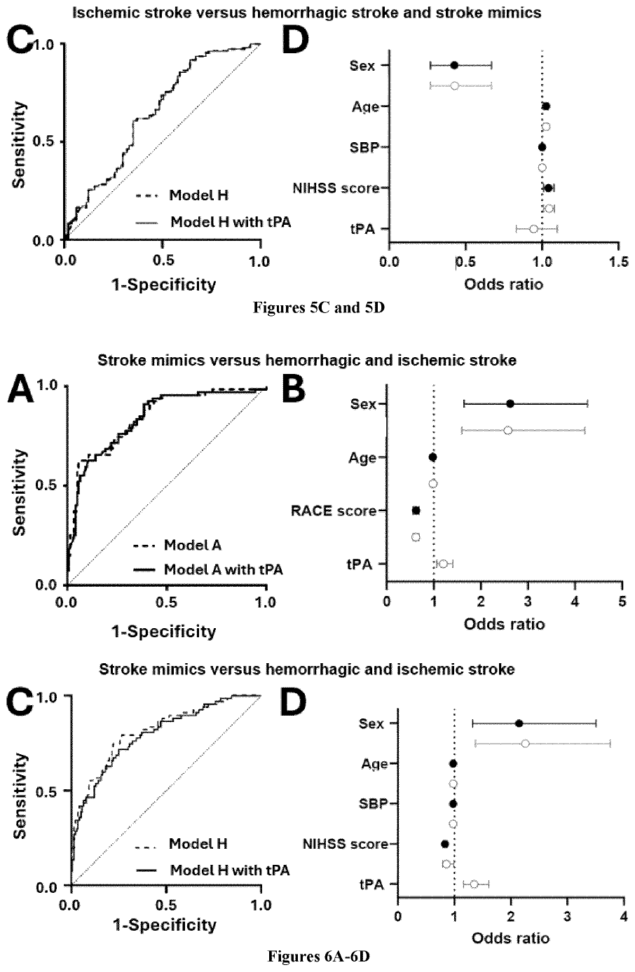

We then tested if our models were able to discriminate ischemic strokes from hemorrhagic strokes and stroke mimics in model A. Among these included variables, sex (OR 0.5, 95% CI [0.32 to 0.78]), age (OR 1.02, 95% CI [1 to 1.03]), and RACE score (OR 1.28, 95% CI [1.17 to 1.40]) demonstrated a significant association with the diagnosis of ischemic stroke when B compared to other types of patients (FIG. 5B and data not shown). The accuracy of the model, excluding tPA as a biomarker, was 0.71, 95% CI [0.65 to 0.76], and was not modified with the inclusion of tPA levels as a parameter (0.71, 95% CI [0.66 to 0.76] p=0.3818; FIG. 5A and data not shown).

In model H, sex (OR 0.43, 95% CI [0.27 to 0.67]), age (OR 1.03, 95% CI [1.01 to 1.04]) and baseline NIHSS (OR 1.04, 95% CI [1.01 to 1.08]) are the clinical parameters with significant impact (FIG. 5D and data not shown). As for model A, the discrimination of ischemic stroke compared to other patients was not increased by the inclusion of circulating tPA levels as a biomarker in the model (0.65, 95% CI [0.59 to 0.70] without tPA, and 0.65, 95% CI [0.59 to 0.70] with tPA; p=0.6072; FIG. 5C and data not shown).

### Identification of Stroke Mimics Within the Cohort

We finally tested if the inclusion of plasma tPA levels in the model increased the discrimination accuracy of stroke mimic against the rest. In model A, sex (OR 2.58, 95% CI [1.61 to 4.2]), age (OR 0.98, 95% CI [0.97 to 0.99]), RACE score (OR 0.62, 95% CI [0.58 to 0.67]), and tPA (OR 1.2, 95% CI [1.07 to 1.39]) were the clinical parameters with significant impact (FIG. 6B and data not shown). The accuracy of the model, excluding tPA as a biomarker, was 0.84, 95% CI [0.79 to 0.88], and was not modified with the inclusion of tPA levels as a parameter (0.84, 95% CI [0.80 to 0.89] p=0.2518; FIG. 6A and data not shown).

In model H, SBP (OR 0.98, 95% CI [0.97 to 0.99]), NIHSS (OR 0.82, 95% CI [0.79 to 0.85]) and tPA levels (OR 1.35, 95% CI [1.16 to 1.60]) exhibited a significant relationship with the clinical categories (i.e. mimics or not) (FIG. 6D and data not shown). Yet, the accuracy of Model H was not modified by the addition of tPA (0.79, 95% CI [0.74 to 0.84] Vs 0.81, 95% CI [0.77 to 0.86]; p=0.0726; FIG. 6C and data not shown).

### Discussion

We demonstrate that, in contrast to patients with ischemic stroke and stroke mimics, individuals suffering from hemorrhagic stroke exhibit lower levels of tPA. Thus, plasma tPA concentration is significantly higher in ischemic patients compared to hemorrhagic patients (FIGS. 1A to 1D).

We have developed two models to evaluate the potential of plasma tPA as a biomarker for identifying hemorrhagic stroke. The first model, known as “Ambulance” (A), incorporates fundamental parameters that can be assessed by paramedics, including age, gender, and RACE score. The second model, the “Hospital” model (H), employs clinical parameters such as age, gender, SBP (Systolic Blood Pressure), and baseline NIHSS (National Institutes of Health Stroke Scale). In both models, the inclusion of plasma tPA levels enhances the accuracy of diagnosing hemorrhagic stroke, thereby establishing it as a valuable biomarker to rapidly detect this condition in a real setting.

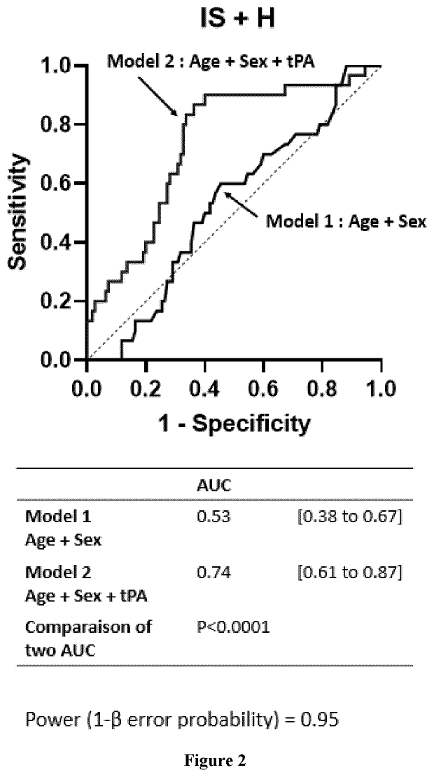

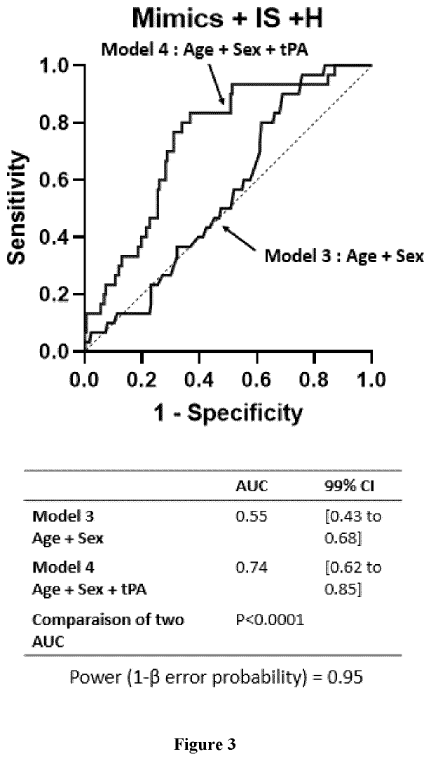

As shown, tPA significantly improves the predictive model for the diagnosis of hemorrhagic stroke compared to ischemic stroke (FIG. 2) or compared to mimic stroke (FIG. 3).

To conclude, dosing the tPA would make it possible to discriminate between hemorrhagic and ischemic stroke, and therefore allows an earlier diagnosis.

### REFERENCES

Throughout this application, various references describe the state of the art to which this invention pertains. The disclosures of these references are hereby incorporated by reference into the present disclosure.

1. A method for discriminating at an early stage an hemorrhagic stroke from an ischemic stroke or a mimic stroke in a subject in need thereof and treating the subject, comprising i) determining in a blood sample obtained from the subject a level of tissue-type plasminogen activator (tPA),
determining that the level of tissue-type plasminogen activator (tPA) is higher than a reference value and that the subject has had or is at risk of developing an ischemic stroke or a mimic stroke, and
treating the subject with a therapeutically effective amount of tPA and/or a classical stroke treatment; or
determining that the level of tissue-type plasminogen activator (tPA) is lower than the reference value and that the subject has had or is at risk of developing a hemorrhagic stroke, and
treating the subject by performing a surgical procedure to remove accumulated blood and drain cerebrospinal fluid.

2. (canceled)

3. The method according to claim 1, wherein the blood sample is a plasma sample or a serum sample.

4. The method according to claim 1, wherein total tPA is measured.

5. The method according to claim 4, wherein the total tPA is measured on citrated plasma.

6. The method according to claim 1, wherein the early stage corresponds to a stage at which the subject had not yet received any treatment or therapeutic care.

7. A method for treating an ischemic stroke in a subject in need thereof comprising administering to the subject a therapeutically effective amount of tPA and/or a classical stroke treatment.

8. (canceled)

9. (canceled)

10. The method according to claim 1, wherein the classical stroke treatment is administration of direct-acting oral anticoagulants (DOACs), anticoagulants, antiplatelet drugs, statins, or blood pressure drugs.

11. (canceled)

12. A kit for use in the method for assessing or diagnose or classify the risk of having or developing a stroke at early stage the said kit comprising a reagent that specifically reacts with tPA.

13. The kit according to claim 12 wherein the stroke is an ischemic stroke.

14. (canceled)
